(19) United States
(12) Patent Application Publication
(10) Pub. No.: US 2002/0042131 A1
(43) Pub. Date: Apr. 11, 2002

(54) Cryopreservation method using cryoprotective composition of propanediol and a vehicle solution

(75) Inventors: Kelvin G.M. Brockbank, Charleston, SC (US); Michael J. Taylor, Mount Pleasant, SC (US); Lia Hanson Campbell, Mount Pleasant, SC (US)

Correspondence Address: Oliff & Berridge PLC, P.O. Box 19928, Alexandria, VA, 22320, US

(73) Assignee: ORGAN RECOVERY SYSTEMS, CHARLESTON, SC

(21) Appl. No.: 09/876,228

(22) Filed: Jun. 8, 2001

Related U.S. Application Data
(60) Provisional application No. 60/210,459, filed on Jun. 9, 2000.

(51) Int. Cl. A01N 1/00; A01N 1/02; C12N 5/00; C12N 5/02

(52) U.S. Cl. 435/374

(57) ABSTRACT

A serum-free method of cryopreserving cells or tissues that includes bringing the cells or tissues into contact with a cryopreservation composition containing propanediol and a vehicle solution such as EuroCollins solution, and subsequently reducing the temperature of the cells to a cryopreservation temperature, for example of at least −80° C.

### BACKGROUND OF THE INVENTION

1. Field of Invention

This invention relates to particular cryoprotectant solutions and methods of cryopreservation utilizing such cryoprotectant solutions. In particular, the invention relates to the use of a cryopreservation composition comprised of propanediol and a vehicle solution in cryopreservation methods.

2. Description of Related Art

Cryobiology may be defined as the study of the effects of temperatures of lower than normal physiologic ranges upon biologic systems. During the past half-century the fundamentals of the science of cryobiology have evolved to the point where low temperatures are now used extensively as a means to protect and preserve biological systems during enforced periods of ischemia and hypoxia. In practice, preservation is achieved using either hypothermia without freezing, or cryopreservation in which the aqueous system sustains a physical phase change with the formation of ice. Survival of cells from the rigors of freezing and thawing in cryopreservation procedures is only attained by using appropriate cryoprotective agents (CPAs) and in general, these techniques are applicable to isolated cells in suspension or small aggregates of cells in simple tissues. More complex tissues and organs having a defined architecture are not easily preserved using conventional cryopreservation techniques, which is principally due to the deleterious effects of ice formation in an organized multicellular tissue. Simply freezing cells or tissues results in dead, nonfunctional materials upon thawing.

The modern era of cryobiology really began with the discovery of the cryoprotective properties of glycerol as reported by Polge et al., “Revival of Spermatazoa After Vitrification and Dehydration at Low Temperatures,” Nature, 164:666 (1949). Subsequently, Lovelock et al., “Prevention of Freezing Damage to Living Cells by Dimethyl Sulfoxide,” Nature, 183:1394 (1959), discovered that dimethyl sulfoxide was also a cryoprotectant, and despite the wide range of compounds now known to exhibit cryoprotective properties, it is still the most widely used compound to date.

A review of the principles of cryobiology can be found in Brockbank, Principles of Cryopreserved Venous Transplantation, Chapter 10, “Essentials of Cryobiology” (1995). A basic principle of cryobiology is that the extent of freezing damage depends upon the amount of free water in the system and the ability of that water to crystallize during freezing. Many types of isolated cells and small aggregates of cells can be frozen simply by following published procedures, but obtaining reproducible results for more complex tissues requires an understanding of the major variables involved in tissue cryopreservation. Major variables involved in tissue freezing include (1) freezing-compatible pH buffers, (2) cryoprotectant choice, concentration and administration, (3) cooling protocol, (4) storage temperature, (5) warming protocol and (6) cryoprotectant elution.

Many cryoprotectants have been discovered. See, for example, Brockbank, supra. Cryoprotectant selection for cryopreservation is usually restricted to those that confer cryoprotection in a variety of biological systems. On occasion, combinations of cryoprotectants may result in additive or synergistic enhancement of cell survival. Comparison of chemicals with cryoprotectant properties reveals no common structural features. These chemicals are usually divided into two classes: (1) intracellular cryoprotectants with low molecular weights that penetrate cells, and (2) extracellular cryoprotectants with relatively high molecular weights (greater than or equal to sucrose (342 daltons)) which do not penetrate cells. Intracellular cryoprotectants, such as glycerol and dimethyl sulfoxide at concentrations from 0.5 to 3 molar, are effective in minimizing cell damage in many slowly frozen biological systems. Extracellular cryoprotective agents such as polyvinylpyrrolidone or hydroxyethyl starch are often more effective at protecting biological systems cooled at rapid rates.

While a variety of factors are known to influence the survival of cells during cryopreservation, the role of the vehicle solution for the cryoprotective agents (CPAs) of the cryoprotective composition is often not considered. It is generally assumed that conventional culture media used to nurture cells at physiological temperatures will also provide a suitable medium for exposure at low temperatures. However, in tissue and organ preservation, maintenance of the ionic and hydraulic balance in cells during hypothermia can be better controlled by using solutions designed to physically restrict these temperature induced imbalances.

What is still desired are improved cryoprotective compositions that increase cell viability during cryopreservation.

U.S. Pat. Nos. 5,217,860 and 5,962,214 describe methods for introducing vitrifiable concentrations of cryoprotective agents into isolated organs or tissues in preparation for cryopreservation and for removing these agents from the organs and tissues after cryopreservation in preparation for transplantation of the organs or tissues. As the CPA, a combination of propanediol, DMSO and formamide is described. As the vehicle solution, preferred solutions include UW solutions, Renal Preservation Solution 2 (RPS-2) solution and EuroCollins solution. These compositions are thus described to be used in vitrification of organs for transplantation. As described in these patents, vitrification is a form of cryopreservation, but involves cooling without freezing (i.e., without formation of ice crystals). Vitrification also requires the use of higher concentrations of CPAs than cryopreservation methods that allow freezing to occur. During vitrification, an “arrested liquid” state known as a ‘glass’ is achieved. In the present invention, cryopreservation with freezing, not vitrification, is being performed.

### SUMMARY OF THE INVENTION

It is therefore one object of the present invention to provide a cryopreservation composition that effectively protects cells during cryopreservation and achieves increased cell viability upon warming from a frozen state.

It is still a further object of the present invention to provide a cryopreservation composition that is capable of obtaining consistent and reproducible results in cryopreserving cells, tissues and organs.

These and other objects are achieved by the present invention, which relates to the use of a synergistic combination of propanediol as a cryoprotective agent (CPA) in a vehicle solution, for example EuroCollins solution as the preferred vehicle for the CPA, in cryopreserving living cells.

In the invention, cells to be cryopreserved are protected against the effects of cryopreservation by bringing the cells into contact with a cryopreservation composition containing propanediol in a vehicle solution such as EuroCollins solution or a suitable variant, and subsequently reducing the temperature of the cells to the cryopreservation temperature.

Also in the invention, the cryopreservation composition may include at least one natural or synthetic ice growth control molecule, such as an anti-freeze protein.

### BRIEF DESCRIPTION OF THE DRAWINGS

FIG. 1 is a flow chart summarizing the cryopreservation procedure utilized in obtaining the results summarized in this application.

FIG. 2 is a plot of relative cell viability of smooth muscle cells after exposure to varying concentrations of dimethyl sulfoxide (DMSO) in (1) EuroCollins and (2) UHK-CV as vehicle solutions.

FIG. 3 is a plot of relative cell viability of corneal endothelial cells after exposure to varying concentrations of dimethyl sulfoxide (DMSO) in (1) EuroCollins and (2) UHK-CV as vehicle solutions.

FIG. 4 is a plot of relative cell viability of smooth muscle cells after exposure to varying concentrations of propanediol in (1) EuroCollins and (2) UHK-CV as vehicle solutions.

FIG. 5 is a plot of relative cell viability of corneal endothelial cells after exposure to varying concentrations of propanediol in (1) EuroCollins and (2) UHK-CV as vehicle solutions.

FIG. 6 is a plot of relative cell viability of smooth muscle cells after freezing using cryoprotective compositions of dimethyl sulfoxide (DMSO) in (1) EuroCollins and (2) UHK-CV as vehicle solutions.

FIG. 7 is a plot of relative cell viability of corneal endothelial cells after freezing using cryoprotective compositions of dimethyl sulfoxide (DMSO) in (1) EuroCollins and (2) UHK-CV as vehicle solutions.

FIG. 8 is a plot of relative cell viability of vascular endothelial cells after freezing using cryoprotective compositions of dimethyl sulfoxide (DMSO) in (1) EuroCollins and (2) UHK-CV as vehicle solutions.

FIG. 9 is a plot of relative cell viability of smooth muscle cells after freezing using cryoprotective compositions of propanediol in (1) EuroCollins and (2) UHK-CV as vehicle solutions.

FIG. 10 is a plot of relative cell viability of corneal endothelial cells after freezing using cryoprotective compositions of propanediol in (1) EuroCollins and (2) UHK-CV as vehicle solutions.

FIG. 11 is a plot of relative cell viability of vascular endothelial cells after freezing using cryoprotective compositions of propanediol in (1) EuroCollins and (2) UHK-CV as vehicle solutions.

### DETAILED DESCRIPTION OF PREFERRED EMBODIMENTS

Through extensive studies, the present inventors have found that the selection of cryoprotective agent (CPA), its concentration and the nature of the vehicle solution are all important variables that can impact the outcome of cryopreservation. Further, it has been found that these variables often do not act in a predictable manner, and thus the selection of these variables may have to be selected through experimentation for cryopreservation of a given cell line. One of the surprising results of these studies has been the discovery that the selection of the vehicle carrier for the cryoprotective composition can significantly impact the survival of cells during cryopreservation, and varies with the nature of the CPAs employed and/or cell type cryopreserved.

Through these studies, the inventors have found a surprising ability of a cryoprotective composition comprised of propanediol as the CPA and a vehicle solution, most preferably EuroCollins solution as the vehicle carrier solution, to significantly increase cell viability during cryopreservation.

Cryopreservation, i.e., the preservation of cells by freezing, in the present invention may be effected in any conventional manner. By “freezing” as used herein is meant temperatures below the freezing point of water, i.e., below 0° C. Cryopreservation typically involves freezing cells to temperatures well below freezing, for example to −130° C. or less. The cryopreservation temperature should be less than −20° C., more preferably −80° C. or less, most preferably −130° C. or less.

The cells to be cryopreserved using the cryoprotective composition of the invention may be in suspension, may be attached to a substrate, etc., without limitation.

In the method of the invention, the cells to be protected during cryopreservation are first brought into contact with the cryopreservation composition. By being brought into contact with the cryopreservation composition is meant that the cells are made to be in contact in some manner with the cryopreservation composition so that during the reduction of temperature to the cryopreservation temperature, the cells are protected by the cryopreservation composition. For example, the cells may be brought into contact with the cryopreservation composition by filling the appropriate wells of a plate to which the cells to be protected are attached, by suspending the cells in a solution of the cryopreservation composition, etc.

The cryopreservation composition of the invention must contain propanediol as the CPA. By “propanediol” it is intended to encompass any propanediol, including, for example, 1,2-propanediol and 1,3-propanediol, as well as mixtures of different propanediol isomers. Preferably, 1,2-propanediol is the propanediol material. Preferably, propanediol is used as the CPA without the use of dimethyl sulfoxide (DMSO) or formamide in the cryoprotective composition.

Preferably, the propanediol is present in the cryopreservation composition in an amount of from, for example, about 0.05M to about 6.0M, more preferably from about 0.5M to about 4.0M, most preferably from about 0.5M to about 3.0M.

The cryopreservation composition also includes a vehicle carrier solution. Most preferably, EuroCollins solution is selected as the vehicle carrier. EuroCollins solution is a well known, commercially available solution comprised of glucose, potassium phosphate monobasic and dibasic, sodium bicarbonate and potassium chloride. The concentrations of the vehicle solution components may be modified from the standard EuroCollins formulation, but should preferably still comprise suitable concentrations of the electrolytes and sugars of at least sodium, potassium, chloride, phosphate monobasic, phosphate dibasic, bicarbonate and glucose. The following Table summarizes amounts of these materials in EuroCollins solution and suitable ranges for variant vehicle solutions.

| | EuroCollins | Variant Vehicle |
|---|---|---|
| Component | (mM) | Solutions (mM) |
| Sodium (Na⁺) | about 10 | about 10 to about 160 |
| Potassium (K⁺) | about 115 | about 3 to about 150 |
| Chloride (Cl⁻) | about 15 | about 15 to about 150 |
| Phosphate monobasic (H₂PO₄⁻) | about 15 | about 1 to about 25 |
| Phosphate dibasic (HPO₄²⁻) | about 42.5 | about 1 to about 50 |
| Bicarbonate (HCO₃) | about 10 | about 5 to about 30 |
| Glucose | about 194 | about 5 to about 1,000 |

The foregoing materials may be suitably combined in any manner, for example as potassium phosphate monobasic, potassium phosphate dibasic, sodium bicarbonate, potassium chloride, etc., for example as in EuroCollins.

The cryopreservation composition may be made by simple addition, for example by mixing, of the CPA to the vehicle carrier solution.

In a still further embodiment of the invention, the cryopreservation composition also includes a natural or synthetic ice growth control molecule such as an anti-freeze protein/peptide (AFP). AFPs also include anti-freeze glycoproteins (AFGPs) and insect anti-freeze, or “thermal hysteresis” proteins, (THPs). Naturally occurring AFPs are believed to be able to bind to the prism face of developing ice crystals, thereby altering their formation. For the fishes and insects in which these proteins occur, it means a depression of their freezing point so they are able to survive under conditions that would normally cause their body fluids to freeze. Any of the well-known AFPs may be used in the present invention in this regard. See, for example, Sicheri and Yang, Nature, 375:427-431, (1995), describing eight such proteins. Most preferably, the AFP may be, for example, AFPI (AFP type I), AFPIII (AFP type III) and/or AFGP. The AFPs may be present in the cryopreservation composition in an amount of from, for example, 0.01 to 1 mg/mL, more preferably 0.05 to 0.5 mg/mL, of composition, for each AFP present.

Once the cells have been contacted with the cryopreservation composition, the cells may then be frozen for cryopreservation. The cryopreservation and subsequent warming of cells may be conducted in any manner, and may utilize any additional materials, well known in the art. Preferred embodiments are described in the following discussion and the Examples set forth below.

The cooling (freezing) protocol for cryopreservation in the present invention may be any suitable type. Many types of cooling protocols are well known to practitioners in the art. Most typically, the cooling protocol calls for continuous rate cooling from the point of ice nucleation to −80° C., with the rate of cooling depending on the characteristics of the cells/tissues being frozen as understood in the art (again, see Brockbank, supra). The cooling rate may be, for example, about −0.1° C. to about −10° C. per minute, more preferably between about −1° C. to about −2° C. per minute. Once the cells are cooled to about −80° C. by this continuous rate cooling, they can be transferred to liquid nitrogen or the vapor phase of liquid nitrogen for further cooling to the cryopreservation temperature, which is below the glass transition temperature of the freezing solution (typically −130° C. or less).

Once cryopreserved, the cells will subsequently be rewarmed for removal of the cryopreserved cells from the cryopreserved state. The warming protocol for taking the cells out of the frozen state may be any type of warming protocol, which are well known to practitioners in the art. Typically, the warming is done in a one-step procedure in which the cryopreserved specimen is placed into a water bath (temperature of about 37-42° C.) until complete rewarming is effected. More rapid warming is also known. Warming may be done to room (ambient) temperature or higher, typically to at least 25° C., more typically to at least 37° C.

The cryopreserved cells, particularly cryopreserved cells fixed to a substrate, may also be warmed by way of the methods described in co-pending U.S. application Ser. No. 09/835,819 filed on Apr. 17, 2001, entitled “Novel Warming Method of Cryopreserved Specimens,” incorporated herein by reference in its entirety. These methods include a two-step warming protocol, with or without the use of a heat sink.

In a most preferred embodiment of the present invention, a cryopreservation composition of propanediol and EuroCollins solution is used to cryopreserve cells selected from the group consisting of smooth muscle cells, for example a smooth muscle cell line A10 derived from rat thoracic aorta, and endothelial cells, for example endothelial cell lines derived from bovine corneal epithelium (BCE) or bovine pulmonary artery endothelium (CPAE).

The following examples illustrate the surprising ability of the cryopreservative composition of the present invention to achieve excellent cell viability after cryopreservation at very low temperatures, for example temperatures of −130° C. or less.

### EXAMPLES

In the Examples, UHK-CV identifies a solution of the type described in application Ser. No. 09/628,311, filed Jul. 28, 2000, incorporated herein by reference in its entirety.

Two cell types, a smooth muscle cell line (A10) derived from rat thoracic aorta and an endothelial cell line (BCE) derived from bovine corneal endothelium, were exposed to a range of concentrations of either dimethyl sulfoxide (DMSO) or 1,2-propanediol (PD), for 10 minutes at 4° C. Elution of the CPAs was accomplished using mannitol as an osmotic buffer at 4° C. Additional groups of cells were treated similarly except they were frozen and thawed in the presence of the various preservation solution/CPA combinations. After rewarming to 37° C. and resuspension in culture medium (DMEM/10% Fetal Calf Serum), all groups of cells were assessed for metabolic activity using the non-toxic indicator Alamar Blue (Trek Diagnostics).

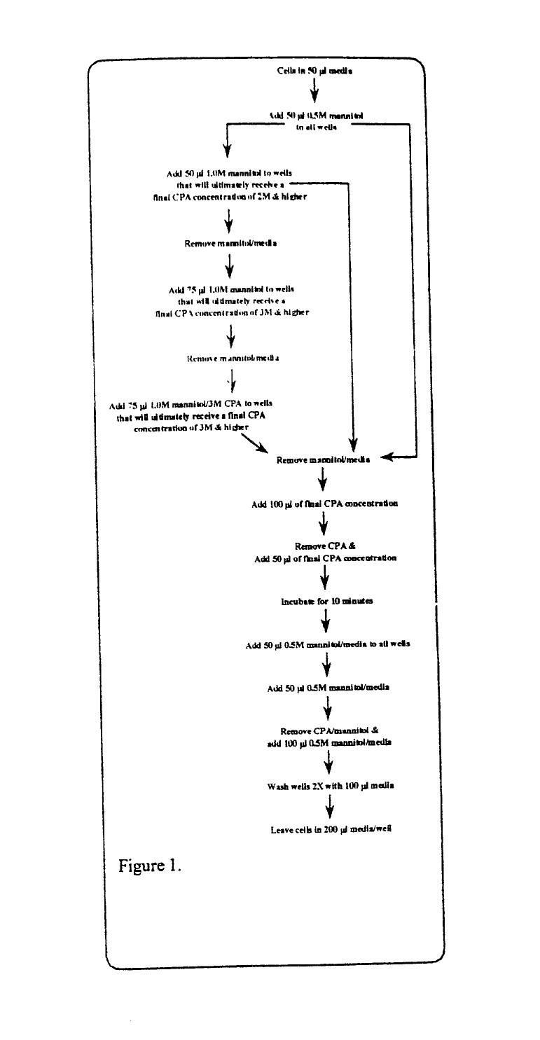

The assay was performed on ice according to FIG. 1 and all solutions used were pre-cooled on ice. Mannitol was used throughout the assay as a non-permeating osmotic buffer. All addition/removal steps in the protocol were performed at five minute intervals unless otherwise noted in FIG. 1. Cell density for the cytotoxicity assay was 1×10⁴ cells/well.

After cells were allowed to recover to physiological temperature for one hour, a volume of 20 μl of Alamar Blue was added to wells containing 1-2×10⁴ cells in 200 μl of media. The plate was incubated at 37° C. for three hours before being read in a fluorescent microplate reader (Fmax fluorescent microplate reader by Molecular Dynamics). Fluorescence from Alamar Blue was determined using an excitation wavelength of 544 nm and an emission wavelength of 590 nm.

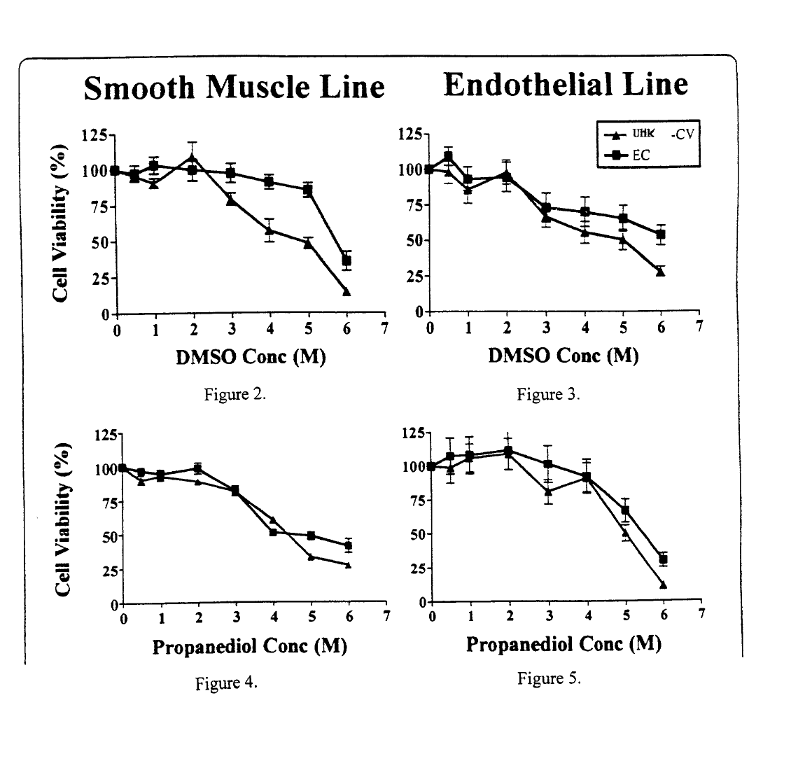

The results are summarized in FIGS. 2-5, summarizing the cell viability after exposure to varying concentrations of cryoprotectants. Two cell types, A10 cells (FIGS. 2 and 4) and BCE cells (FIGS. 3 and 5) were exposed to varying concentrations (0-6M) of dimethyl sulfoxide (DMSO) (FIGS. 2 and 3) and 1,2 propanediol (FIGS. 4 and 5) in either EuroCollins (EC) or UHK-CV. The cells were exposed to the CPAs using the protocol shown in FIG. 1. Data was normalized to the vehicle solution alone (OM concentration) and is the mean (±SEM) of 12 replicates.

The freezing assay is performed using the same steps as illustrated in FIG. 1 for the cytotoxicity assay with the following modifications. Cell density for the freezing assay was 2×10⁴ cells/well. After the final addition of CPA, the plate was cooled at −1.0° C./min to −80° C., then stored at −135° C. (overnight) in a LN₂ storage freezer. The next day, the plate was removed from the freezer and allowed to equilibrate in a −20° C. freezer for thirty minutes. Following equilibration, the plate was removed from the freezer and thawed rapidly in a 37° C. water bath. During this period (˜1-2 minutes), 0.5M mannitol media warmed to 37° C. was added to the wells. The plate was then immediately removed from the water bath and put on ice. The remainder of the removal steps shown in the flow chart were performed followed by assessment of viability using Alamar Blue.

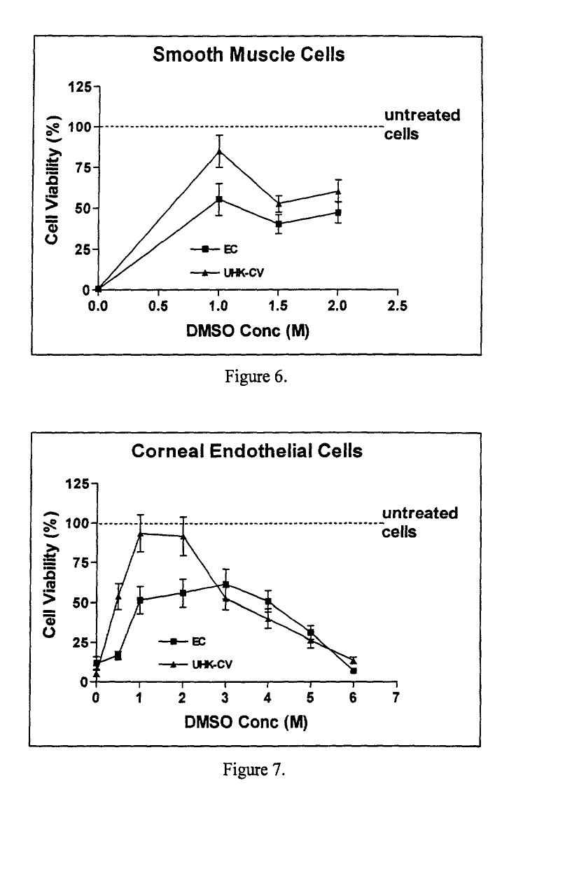

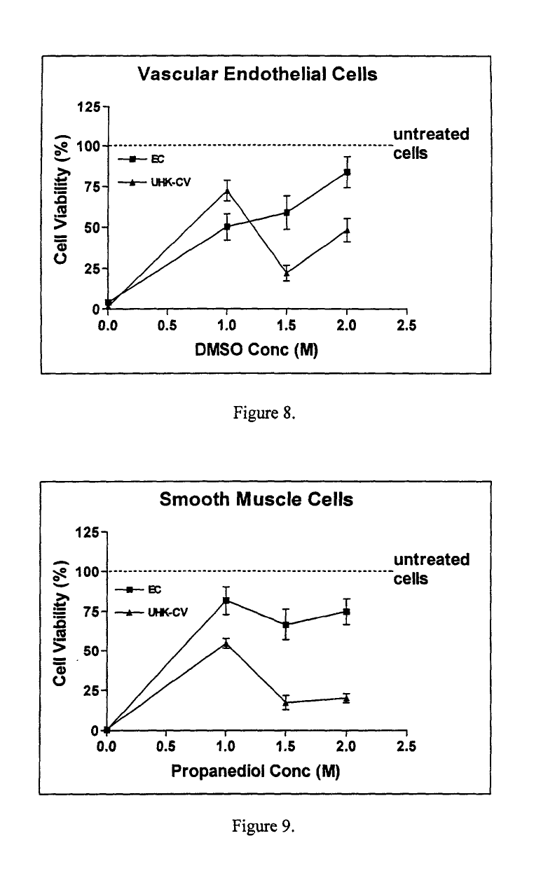

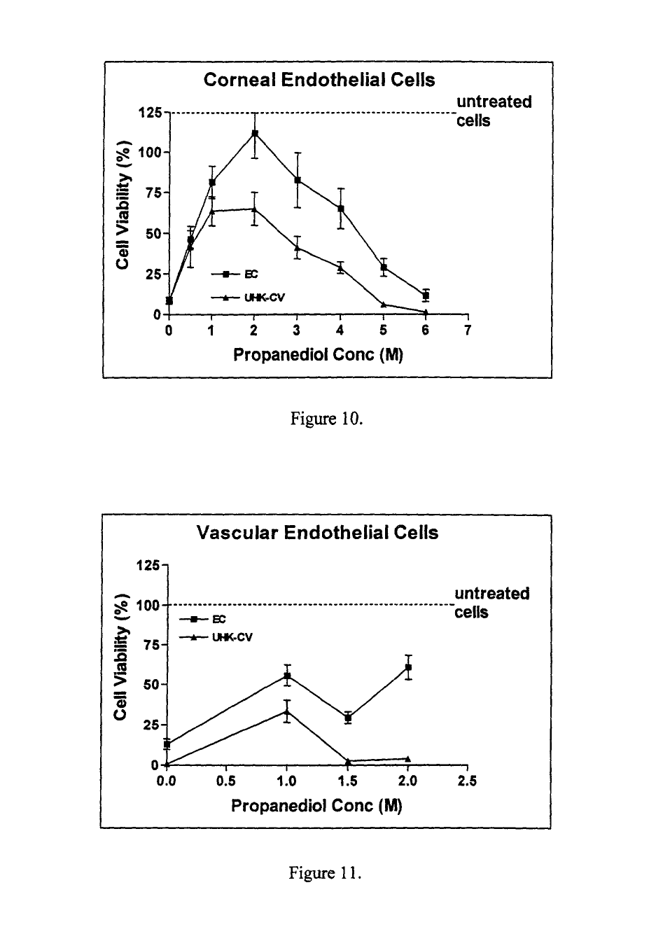

The results are summarized in FIGS. 6-11, summarizing the cell viability after freezing with varying concentrations of DMSO (FIGS. 6-8) and propanediol (FIGS. 9-11). A10 cells (FIGS. 6 and 9), BCE cells (FIGS. 7 and 10) and CPAE cells (FIGS. 8 and 11) were cryopreserved in DMSO or propanediol (0-6M) in either EC or UHK-CV following the freezing protocol described above. Data was normalized to untreated cells and is the mean (±SEM) of 12 replicates.

As seen particularly from the results summarized in FIGS. 9-11, and as compared to the results in FIGS. 6-8, a cryopreservation composition of propanediol and EuroCollins solution achieves an unexpected result in terms of cell viability following freezing to very low cryopreservation temperatures (for example, −130° C. and less) as compared to the use of a cryopreservation composition of DMSO in EuroCollins solution or in UHK-CV and as compared to the use of propanediol in UHK-CV. These results are quite unexpected and indicate a surprising, previously unknown synergistic effect associated with the use of the cryopreservation composition of the present invention, particularly with respect to the cryopreservation of smooth muscle cells and endothelial cells.

While this invention has been described in conjunction with specific embodiments thereof, it is evident that many alternatives, modifications and variations will be apparent to those skilled in the art. Accordingly, the preferred embodiments of the invention as set forth herein are intended to be illustrative only, and not limiting. Various changes may be made without departing from the spirit and scope of the invention as defined in the following claims.

What is claimed is:

1. A method of cryopreserving cells or tissues, comprising bringing the cells into contact with a cryopreservation composition containing propanediol and a vehicle solution containing at least sodium, potassium, chloride, phosphate monobasic, phosphate dibasic, bicarbonate and glucose, and free of dimethyl sulfoxide and formamide, and subsequently reducing the temperature of the cells or tissues to a cryopreservation temperature.

2. A method according to claim 1, wherein the propanediol is 1,2propanediol.

3. A method according to claim 1, wherein the propanediol compound is present in the cryopreservation composition in an amount of from about 0.05M to about 6.0M.

4. A method according to claim 1, wherein the vehicle solution is EuroCollins solution.

5. A method according to claim 1, wherein the cryopreservation temperature is −80° C. or less.

6. A method according to claim 1, wherein the cells or tissues are cells selected from the group consisting of smooth muscle cells and endothelial cells.

7. A method according to claim 1, wherein the cryopreservation composition further contains at least one ice growth control molecule of a natural antifreeze protein or a synthetic compound.

8. A method according to claim 1, wherein the method further comprises warming the cells or tissues from the cryopreservation temperature to a temperature of at least 25° C.

9. A method of cryopreserving cells or tissues, comprising bringing the cells or tissues into contact with a cryopreservation composition comprised of about 0.05M to about 6.0M propanediol and a vehicle solution containing at least sodium, potassium, chloride, phosphate monobasic, phosphate dibasic, bicarbonate and glucose, and subsequently reducing the temperature of the cells or tissues to −80° C. or less.

10. A method according to claim 9, wherein the propanediol is 1,2-propanediol.

11. A method according to claim 9, wherein the vehicle solution is EuroCollins solution.

12. A method according to claim 9, wherein the cryopreservation temperature is −130° C. or less.

13. A method according to claim 9, wherein the cells or tissues are cells selected from the group consisting of smooth muscle cells and endothelial cells.

14. A method according to claim 9, wherein the cryopreservation composition further contains at least one ice growth control molecule of a natural antifreeze protein or a synthetic compound.

15. A method according to claim 9, wherein the method further comprises warming the cells or tissues from the cryopreservation temperature to a temperature of at least 25° C.

16. A method of cryopreserving cells, comprising bringing cells selected from the group consisting of smooth muscle cells and endothelial cells into contact with a cryopreservation composition comprised of propanediol and a vehicle solution containing at least sodium, potassium, chloride, phosphate monobasic, phosphate dibasic, bicarbonate and glucose, and subsequently reducing the temperature of the cells or tissues to a cryopreservation temperature.

17. A method according to claim 16, wherein the propanediol compound is present in the cryopreservation composition in an amount of from about 0.05M to about 6.0M.

18. A method according to claim 16, wherein the cryopreservation temperature is −80° C. or less.

19. A method according to claim 16, wherein the method further comprises warming the cells or tissues from the cryopreservation temperature to a temperature of at least 25° C.

20. A cryopreservation composition comprising propanediol and a vehicle solution containing at least sodium, potassium, chloride, phosphate monobasic, phosphate dibasic, bicarbonate and glucose, and free of dimethyl sulfoxide and formamide.

21. A cryopreservation composition according to claim 20, wherein the vehicle solution is EuroCollins solution.
